(19) United States
(12) Patent Application Publication
(10) Pub. No.: US 2008/0077002 A1
(43) Pub. Date: Mar. 27, 2008

(54) Compounds and Methods for Combined Optical-Ultrasound Imaging

(75) Inventors: Tim Nielsen, Hamburg (DE); Steffen Weiss, Hamburg (DE); Tobias Schaeffter, Hamburg (DE); Bernhard Gleich, Hamburg (DE); Paul Haaker, Hamburg (DE); Udo Van Stevendaal, Ahrensburg (DE); Peter Mazurkewitz, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/574,747

(22) PCT Filed: Sep. 6, 2005
(86) PCT No.: PCT/IB05/52898

§ 371 Date: Mar. 6, 2007

(30) Foreign Application Priority Data
Sep. 10, 2004 (EP) 04104388.6

(51) Int. Cl. A61B 5/05

(52) U.S. Cl. 600/407

(57) ABSTRACT

The present invention relates to novel methods and compounds for combined opticalultrasound imaging. The compounds of the present invention relate to particles comprising fluorescence donor and acceptor molecules for energy exchange via FRET. The methods of the present invention use ultrasound to modify the distance between donor and acceptor molecules present on the particles, and to consequently modify the fluorescence emitted by the donor and acceptor. The compounds and methods of the present invention are useful in medical or diagnostic imaging.

The invention relates to compounds for use in methods for analytical or diagnostic ultrasound or optical imaging, especially to the provision of contrast agents, methods of using ultrasound or optical imaging, e.g. for analysis of biological tissues or for diagnosis of tissues of patients as well as apparatus for carrying out analytical or diagnostic ultrasound or optical imaging.

Several techniques exist for diagnostic imaging of a body part, including ultrasound imaging and fluorescence imaging. A major problem in fluorescence imaging in turbid media (e.g. tissue) is that spatial resolution is very poor due to strong scattering of both the excitation light and the emitted fluorescence light. Consequently, the resolution of conventional optical fluorescence tomography is limited to about 1 cm³. Such scattering of the excitation source does not occur when ultrasound is being used. Spatial resolution depends on the ultrasound focus size which is of the order of 1 mm².

Lack of modulation is another problem that is encountered when using light (e.g. fluorescent light) as an imaging tool. It is known to reproduce images by the reconstructing images created by a combination of acoustic waves and illumination, see the book “Acousto-optics” by A. Korpel, Marcel Dekker Inc. 1997. In such methods the change of refractive index caused by acoustic waves is visualised by the effect of the change on refractive index on incident light. However, the change in index caused by acoustic waves is small and the images are of poor quality.

Another method which allows changes in light intensity involves modifying the distance between the partners of a fluorescence donor acceptor pair. The donor molecules absorb excitation light but do not emit fluorescence. If a donor is close to an acceptor, the energy is transferred to the acceptor by fluorescence resonance energy transfer (FRET) or more generally due to direct dipole-dipole interaction and the acceptor emits fluorescence. The fluorescence intensity depends thus on the distance between donor and acceptor. Fluorescence Resonance Energy Transfer FRET is a phenomenon which is strongly dependent on the distance (∝r⁻⁶), between donor and acceptor. The transition from 0% to 100% energy transfer is very sharp, i.e. a high fluorescence modulation can be achieved. FRET has been widely used in biological application for determining the binding between proteins or to study membrane structure or to study interactions between membranes. For these purposes, vesicles were developed which contain a fluorescence donor and/or acceptor for FRET (Wong and Groves (2002) Proc. Natl. Acad. USA 99,14147-14152; John et al. (2002) Biophys. J 83, 1525-1534; Leidy et al (2001) Biophys J. 80, 1819-1828).

Ultrasound microbubble vesicles comprising fluorescent groups are known, e.g. U.S. Pat. No. 6,123,923.

An object of the present invention is to provide any of: an imaging method combining ultrasound and optical imaging, contrast agents for such a method, apparatus for such a method including display of images, the images themselves and/or software for use in the method.

An advantage of the present invention is provision of compositions and methods for fluorescence imaging, such as fluorescent tomography.

An aspect of the present invention relates to compounds, compositions or similar for use in methods which allow the modulation of emitted fluorescence by a contrast medium by means of changing the distance between fluorescence donor and fluorescent acceptor. By changing the distance, the fluorescence can be turned on, modulated, or turned off. The methods and compounds may be used for imaging especially for diagnostic imaging, e.g. as practised on tissue samples, body organs for transplantation, or human or animal patients.

The present invention describes novel compounds, compositions and methods for combined optical-ultrasound imaging. In this combined method, the ultrasound is used for high spatial resolution and the fluorescence detection leads to high sensitivity.

The compounds of the present invention can be particles comprising donor and/or acceptors or donor and/or acceptor groups for energy exchange via FRET.

According to an aspect of the present invention an ultrasound field can be used to switch the compound or composition from a non-fluorescent to a fluorescent state (or vice versa) using for example flexible particles such as vesicular flexible particles, e.g. microbubbles with fluorescence donors and/or fluorescence acceptors.

According to another aspect of the present invention, a particle can be forced to deform or oscillate by an ultrasound field, which may be focussed. This results in a change of the distance between a fluorescence donor and fluorescent acceptor on or in the particle. In a particular embodiment, related to FRET, the transition from 0% to 100% energy transfer is very sharp, due to the strong dependence of FRET on the distance (∝r⁻⁶). Consequently a high fluorescence modulation can be achieved.

Using the methods of the present invention the spatial resolution can be limited by the ultrasound focus size which is on the order of 1 mm³. This is three orders of magnitude better than the resolution of conventional optical fluorescence tomography (≈1 cm³). Accordingly, the use of the particles of the present invention as a contrast agent combines the high sensitivity of fluorescence imaging with the spatial resolution of ultrasound.

The present invention provides methods for molecular imaging based on optical imaging with high sensitivity (comparable to PET), but without radioactive compounds. The present invention also provides methods for obtaining a high spatial resolution in optical fluorescence imaging or tracking such as in tissues or turbid media.

The invention also relates to the use of a particle comprising a fluorescence donor and/or a fluorescence acceptor in the manufacture of a contrast agent for combined optical-ultrasound imaging. The donor and/or acceptor can be attached to the particle, e.g. covalently.

The invention further relates to the use of a particle comprising a fluorescence acceptor and/or a fluorescent donor for the modulation of fluorescent light emission after the application of ultrasound. Both donor and acceptor can be present on the ultrasound particle. Alternatively none or either the acceptor or the donor is present on the particle. The additional donors and or acceptors are administered independently and interact or integrate with the particle. The fluorescence can by generated by FRET but also by other mechanisms of energy transfer such as excited state reactions.

In addition the change in fluorescence emitted by the particles after application of the ultrasound can be recorded.

The invention further relates to a combined optical-ultrasound contrast medium comprising a particle with a fluorescence donor and acceptor. According to one embodiment, the donor and/or acceptor are attached to the particle, e.g. covalently.

The invention further relates to a method for the manufacture of a particle for ultrasound imaging wherein the particle or a compound for said particle is brought in contact with fluorescence donors or acceptors. The donors and acceptors can be added separately, together or consecutively. In addition, the fluorescence donor and/or acceptor can be linked with the ultrasound particle or a compound for such a particle, e.g. covalently.

The invention further relates to a kit of parts for combined optical-ultrasound imaging comprising at least two of the groups selected from an ultrasound source, a monitor for recording fluorescent light and particles having a fluorescence acceptor and/or a fluorescent donor.

The invention further relates to a pharmaceutical composition comprising particles having a fluorescence acceptor and/or a fluorescence donor, said particles further comprising a pharmaceutically active compound.

The invention also relates to a method of providing an image of a body part or tissue of an individual having a contrast medium which comprises particles comprising a fluorescence donor and/or a fluorescence acceptor. This is performed by subjecting the body part or tissue to ultrasound and recording the modulation in fluorescent light emitted by the contrast medium.

The invention also relates to a device for ultrasound imaging wherein the device comprises an ultrasound source and an apparatus for the detection of emitted fluorescent light. The light emission can be locally modulated by focussing an ultrasound beam. The invention relates to an apparatus for combined optical-ultrasound imaging comprising an ultrasound source and a detector for the detection of emitted fluorescent light and further comprising a reconstruction unit for the generation of an image from detected fluorescent light. In addition the apparatus can comprise a means for synchronising the emission of ultrasound and/or the detection of fluorescent light and/or the generation of an image. The apparatus can also Further comprise a connection between the reconstruction unit and the detector for the detection of emitted fluorescent light. Furthermore the apparatus can comprise a connection between the reconstruction unit and the ultrasound source or can further comprise a light source or a recorder for recording ultrasound. The apparatus can also comprise a control unit for controlling the generation of ultrasound and/or recording of ultrasound with the emission of light by the light source and/or the detection of light recorded. Such a light source can emit light of a continuous-wave, of a modulated wave or of a pulsed wave. The ultrasound source can have means for focussing the ultrasound beam to thereby locally modulate light emission from particles with at least a fluorescent acceptor or a fluorescent donor. The ultrasound source can also have means to generate pulses of sound waves or to generate extended sound waves with varying frequencies and/or varying direction.

The present invention also provides a computer based apparatus for executing a reconstruction algorithm of an image of an object from data received from an ultrasound source and detected emitted fluorescent light from a contrast medium which comprises particles comprising a fluorescence donor and/or a fluorescence acceptor, the reconstruction algorithm for the generation of the image from detected fluorescent light comprising a pressure dependent fluorescence model of the contrast medium. The apparatus can comprise means for measuring the concentration of the contrast agent by ultrasound imaging. The ultrasound source can emit sound waves that are pulses and focused to one or more lines or one or more spots.

The present invention also provides a computer based method for executing a reconstruction algorithm of an image of an object from data received from an ultrasound source and detected emitted fluorescent light from a contrast medium which comprises particles comprising a fluorescence donor and/or a fluorescence acceptor, the method comprising reconstructing the image from detected fluorescent light using a pressure dependent fluorescence model of the contrast medium. The method can include measuring the concentration of the contrast agent by ultrasound imaging. The ultrasound source preferably emits sound waves that are pulses and focused to one or more lines or one or more spots.

The present invention also includes a software product comprising code for execution of any of the methods of the present invention when executed on a processing engine. The present invention also includes a machine readable data storage device storing the software product, e.g. diskettes, an optical storage device such as a CD-ROM or a DVD-ROM, a hard disk of a computer, a tape storage device, a memory of a computer, e.g. RAM or ROM.

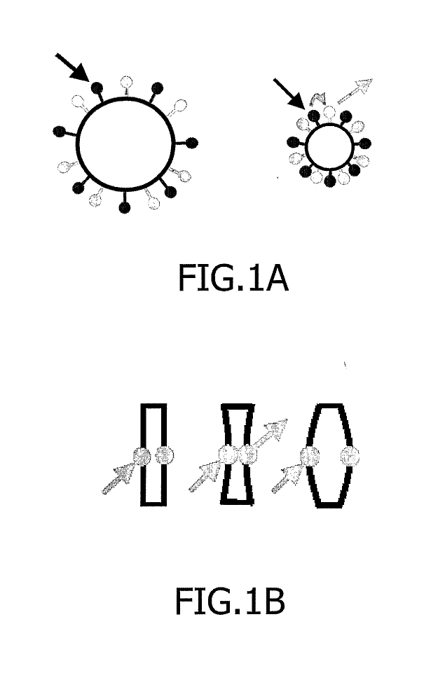

FIG. 1A shows in accordance with an embodiment of the present invention the principle of fluorescence on a compressed vesicular particle. The left panel shows the particle (e.g. vesicle) in a relaxed state: The donor molecule (gray) absorbs energy from the excitation light (black arrow) but the distance between donor and acceptor (gray) is too large for energy transfer to occur. The right panel shows the compressed or deformed state of the particle; herein the energy is transferred from the donor to the acceptor (bent arrow), and acceptor fluorescence (gray arrow) is emitted.

FIG. 1B illustrates an alternative embodiment wherein the particle has a rectangular or rod like shape.

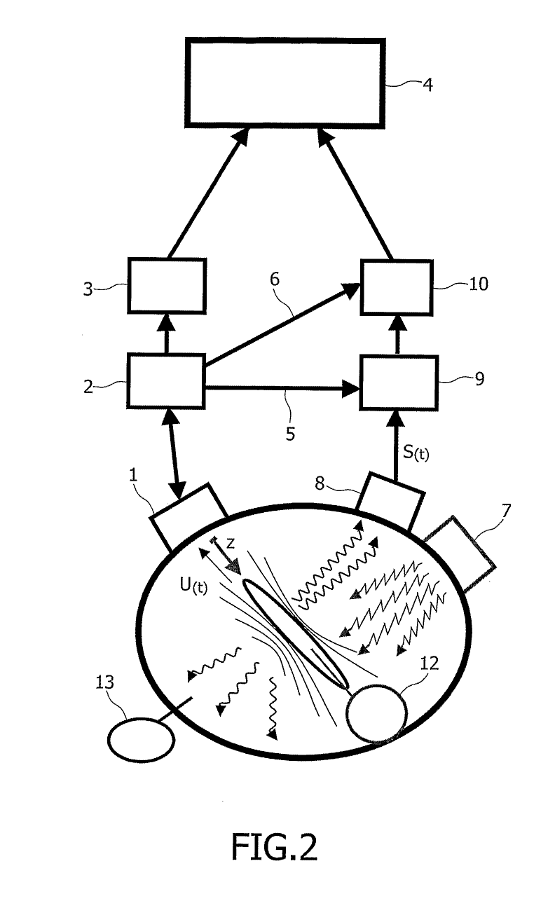

FIG. 2 is a schematic representation of an apparatus for combined ultrasound and fluorescent imaging according to an embodiment of the present invention. 1: US transducer; 2: US generator/receiver; 3: US image reconstructor; 4: display unit; 5: gate signal;6: US envelope signal;7: optical excitation source; 8: fluorescence detector; 9: A/D converter; 10: optical reconstructor; 12: particles with FRET donors and acceptors; 13: body

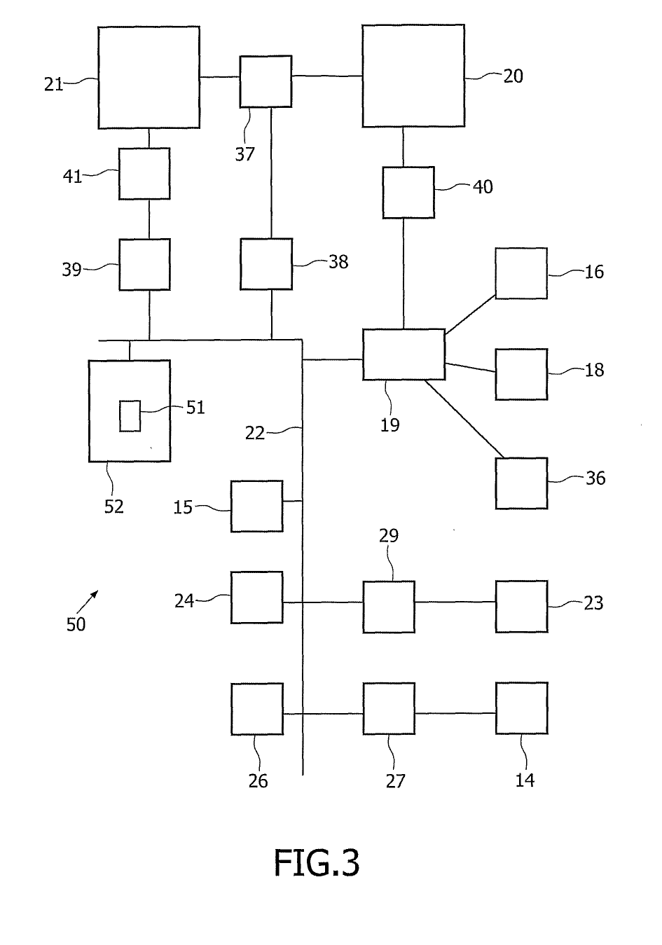

FIG. 3 is a schematic block diagram of a computer-based apparatus for combined ultrasound and fluorescent imaging according to an embodiment of the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In one aspect the present invention relates to particles comprising at least one conjugate or pair of fluorescence donors and acceptors or at least one conjugate of fluorescence donor and acceptor elements of a molecule which is/are arranged in such a way that deformation of the particle brings the donors and acceptors closer together. The donor and acceptor conjugate or molecules may be attached covalently to the particle.

A donor or an acceptor can be any of a molecule, a group of molecules, a complex of the types and examples of donors or acceptor being referred to in the present invention.

According to an embodiment of the present invention the particles of the invention are deformable or flexible. The particles may be globular particles such as vesicles. “Vesicle” refers to an entity which generally has one or more walls or membranes which form one or more internal voids. Vesicles may be formulated, for example, from a stabilizing material such as a lipid, a protein, a polymer, a surfactant and/or a carbohydrate. The lipids, proteins, polymers, surfactants and/or other vesicle forming stabilizing materials may be natural, synthetic or semi-synthetic. The walls or membranes may be concentric or otherwise. The stabilizing compounds may be in the form of one or more monolayers or bilayers. In the case of more than one monolayer or bilayer, the monolayers or bilayers may be concentric. Stabilizing compounds may be used to form a unilamellar vesicle (comprised of one monolayer or bilayer), an oligolamellar vesicle (comprised of about two or about three monolayers or bilayers) or a multilamellar vesicle (comprised of more than about three monolayers or bilayers). The walls or membranes of vesicles may be substantially solid (uniform), or they may be porous or semi-porous. The internal void of the vesicles may be filled with a wide variety of liquid, gaseous or solid materials (or combinations thereof) including, for example, water, oils, fluorinated oils, gases, gaseous precursors, liquids, and fluorinated liquids, if desired, and/or other materials. The vesicles may also comprise a photoactive agent, a bioactive or pharmaceutical compound and/or a targeting ligand, if desired.

Globular particles which are particularly suitable for the compounds and methods of the present invention are preferably biocompatible and/or highly compressible or expandable. Examples are microbubbles. These can be small, 3 to 5 μm diameter, gas-filled spheres that provide their enhancement through several mechanisms linked to their high compressibility when exposed to an ultrasonic pressure field. [de Jong, N. and F. J. T. Cate, in Ultrasonics, 1996. 34(2-5): p. 587-590; Moran, C. M., et al. in. Ultrasound in Medicine & Biology, 2002. 28(6): p. 785-791.]. Currently there are three ultrasound contrast agents approved on the U.S. market. Definity®, marketed by Bristol-Myers-Squibb and developed by Unger at ImaRX, consists of 1.1 to 3.3 micron diameter spheres with a lipid shell and octafluoropropane gas interior. Optison®, marketed by Amersham and originally developed by Mallinckrodt, contains spheres with diameters ranging from 2 to 4.5 microns, albumin shells, and containing octafluoropropane gas. Albunex®, also marketed by Amersham, is a first generation agent similar to Optison® but containing room air. In Europe, there are several approved agents. Sonovue®, marketed by Bracco, is a phospholipid coated sulphur hexafluoride microbubble with a mean size of 2.5 microns. Echovist® and Levovist®), marketed by Schering have been in use for some time and consist of sugar-stabilized room air microbubbles with less-controlled size distributions (>5 μm).

The physical mechanism for ultrasound contrast involves the high compressibility of the gas within the bubble and the physical size of the bubble [de Jong cited supra ; Harvey, C. J., et al., in Advances in Ultrasound. Clinical Radiology, 2002. 57(3): p. 157-177; Calliada, F., et al. in Ultrasound contrast agents: Basic principles. European Journal of Radiology, 1998. 27(2): p. S157-S160.]. At diagnostic imaging frequencies, the microbubbles can undergo oscillations that are many multiples of the resting diameters. This effect is especially exaggerated near the resonance of the gas bubble. By careful choice of the gas within the microbubble and the elastic characteristics of the shell material, the stability of the bubble and its contrast effect can be manipulated. The large-scale oscillations lead to many non-linear effects.

Also liposomes are potentially useful contrast agents for ultrasound imaging. Liposomes have been used for more than 25 years as a potential mechanism for drug delivery. Most liposomes are not echogenic, consisting primarily of fat. Usually liposomes consist of non-gaseous, multi-lamellar acoustically reflective lipids. [Demos, S., et al.,. Journal of the American College of Cardiology, 1999. 33: p. 867-875.] These liposomes are characterised by the presence of many small and irregularly shaped vesicles arranged in a “raspberry-like” appearance. The liposomes are typically smaller than 1 micron in diameter. The usage of liposomes results in an enhanced appearance in ultrasound imaging due to scattering process. Liposomes however have a low stability and half-life and no major mechanical resonance is connected with liposomes.

According to another embodiment of the invention the particles are micellar. Micelle refers to a colloidal entity formulated from lipids. In preferred embodiments, micelles comprise a monolayer, bilayer, or hexagonal H II phase structure (a generally tubular aggregation of lipids in liquid media) see for example U.S. Pat. No. 6,033,645.

Particles with other shapes than globular shaped can be deformed via ultrasound in order to change the distance between fluorescence donor and acceptor molecules which are present on the particle. Non-globular particles which are suitable for the compounds and methods of the present invention are rod-like or Y shaped, tubular or rectangular.

According to another embodiment of the invention the particles are aerogels. Aereogel refers to generally spherical or spheroidal entities which are characterized by a plurality of small internal voids (see for example U.S. Pat. No. 6,106,474). The aerogels may be formulated from synthetic materials (for example, a foam prepared from baking resorcinol and formaldehyde), as well as natural materials, such as carbohydrates (polysaccharides) or proteins.

According to another embodiment of the invention the particles are clathrates. Clathrate refers to a solid, semi-porous or porous particle which may be associated with vesicles. In a preferred form, the clathrates may form a cage-like structure containing cavities which comprise one or more vesicles bound to the clathrate, if desired. A stabilizing material may, if desired, be associated with the clathrate to promote the association of the vesicle with the clathrate. Clathrates may be formulated from, for example, porous apatites, such as calcium hydroxyapatite, and precipitates of polymers and metal ions, such as alginic acid precipitated with calcium salts, see for example U.S. Pat. No. 5,086,620.

In accordance with a method of the present invention the particles are subjected to an ultrasound field, resulting in a deformation and/or oscillation of the particles. Ultrasonic waves are longitudinal compression waves. For longitudinal waves the displacement of the particles in the medium is parallel to the direction of wave motion as opposed to transverse waves for which the displacement is perpendicular to the direction of propagation. Ultrasound refers to any frequency at the high end or above the audible spectrum of the human ear (20 to 20,000 Hz). Medical imaging uses typically frequencies of about 2,5 MHz. In the present invention, lower or higher frequencies can be selected as desired, depending on the type of tissue being examined and the type of particles being used. A commonly used parameter in ultrasound imaging is the mechanical index (=peak refractional or negative pressure divided by the square root of the ultrasound frequency,). The mechanical index is related to the peak negative pressure in the tissue and thus relates to the stiffness of the particles which can be used and still provide enough deformation to achieve an effect used in embodiments of the present invention. Clinical values of the MI are between 1 and 2. In a particular embodiment, globular particles of the present invention can be compressed in volume by a factor of between at least 5, to about 10, 25, 50 or 100, in order to bring fluorescence donor and acceptor molecules into each other's proximity. In another particular embodiment, globular particles of the present invention can be expanded in volume by a factor of between at least 5, to about 10, 25, 50 or 100, in order to move donor and acceptor molecules away from each other.

According to one embodiment of the invention, the fluorescence donors and acceptors on the particles of the present invention exchange energy via FRET (Fluorescence resonance energy transfer). FRET is the transfer of the excited state energy from a donor (D) to an acceptor (A), and can occur when the emission spectrum of the donor (D) fluorophore overlaps the absorption spectrum of the acceptor (A) fluorophore. Thus, by exciting at the absorption maximum of the donor and monitoring the emission at the long wavelength side of the acceptor fluorophore, it is possible to monitor only D and A molecules that are bound and reside within a certain distance, r.

### Thus one can monitor either the quenching of D or enhanced emission of A. The transfer rate, k_{T} in sec⁻¹ is mathematically defined as

k_{T}=(r⁻⁶JK²n⁻⁴λ_{d})×8.71×10²³ (Equation 1)

where r is the D-A distance in Angstrom, J is the D-A overlap integral, K² is the orientation factor, n is the refractive index of the media, and λ_{d} is the emissive rate of the donor. The overlap integral, J, is expressed on the wavelength scale by

$$J = {\int }_{0}^{∞} {F}_{d} ( λ ) {ɛ}_{a} λ ( {λ}^{4} ) d λ \qquad ( Equation 2 )$$

where its units are M⁻¹cm³, F_{d} is the corrected fluorescence intensity of the donor as a function of wavelength λ, and εₐ is the extinction coefficient of the acceptor in M⁻¹ cm⁻¹. Constant terms in equation 2 are generally combined to define the Forster critical distance, Rₒ, which is the distance in angstroms at which 50% transfer occurs. By substitution then, Rₒ can be defined in terms of the overlap integral, J, in Angstrom, as

Rₒ=9,79×10³(K⁻²n⁻⁴ΦJ ^{15/6} (Equation 3)

with Φ_{d} being the quantum yield of the donor. Rₒ and r are related to the transfer efficiency, E by

$$E = \frac{{R}_{0}^{6}}{{R}_{0}^{6} + {r}_{20}^{6}} \qquad ( Equation 4 )$$

which determines the practical distance by which D and A can be separated to obtain a usable signal.

From these equations one can derive that, for high sensitivity, Donor-Acceptor pairs are chosen which have high quantum yields, high J values, and high Rₒ values. For example, Rₒ for the fluorescein/rhodamine pair is about 55 Angstrom. Large values of Rₒ are desired to achieve a measurable signal when molecules containing D and A bind to each other. In practice it is common to use twice as many acceptor as donor molecules if the emission of A is to be used as the readout.

Although the donor and the acceptor are referred to a “pair”, the two “members” of the pair can be the same substance, that is they can be a conjugate comprising two elements of the same molecule. Generally, the two members will be different (e.g., fluorescein and rhodamine). It is possible for one molecule (e.g., fluorescein and rhodamine) to serve as both donor and acceptor; in this case, energy transfer is determined by measuring depolarization of fluorescence. It is also possible for more than two members, e.g. two donors and one acceptor or any other combination.

Reference to either donor or acceptor molecule depends on the function of the molecule in the energy transfer complex. A molecule in a complex is characterised by its physical properties namely absorbing light of a certain wavelength or not, or emitting fluorescence or not. This classifies a molecule as being inactive, fluorescent or quencher. Thus it is possible that a green dye can be a donor for a red dye and can be an acceptor for a blue dye at the same time.

Examples of useful donor-acceptor pairs include NBD (i.e., N-(7-nitrobenz-2-oxa-1,3-diazol-4-yl)) to rhodamine, NBD to fluorescein to eosin or erythrosine, dansyl to rhodamine, and acrdine orange to rhodamine. Examples of suitable commercially available labels capable of exhibiting FRET include fluorescein to tetramethylrhodamine; 4,4-difluoro-5,7-dimethyl-4-bora-3a,4a-diaza-s-indacene-3-propionic acid, succinimidyl ester, which is commercially available, e.g., under the trade designation BODIPY FL from Molecular Probes (Eugene, Oreg.) to 4,4-difluoro-5-phenyl-4-bora-3a,4a-diaza-sindacene-3-propionicacid, succinimidyl ester, which is commercially available, e.g., under the trade designation BODIPY R6G from Molecular Probes; Cy3.5 monofinctional NHS-ester to Cy5.5 monofunctional NHS-ester, Cy3 monofunctional NHS-ester to Cy5 monofunctional NHS-ester, and Cy5 monofunctional NHS-ester to Cy7 monofunctional NHS-ester, all of which are commercially available from Amersham Biosciences (Buckinghamshire, England); and ALEXA FLUOR 555 carboxylic acid, succinimidyl ester to ALEXA FLUOR 647 carboxylic acid, succinimidyl ester, which are commercially available from Molecular Probes.

Other examples of molecules that are used in FRET include the fluorescein derivatives such as 5-carboxyfluorescein (5-FAM), 6-carboxyfluorescein (6-FAM), fluorescein-5-isothiocyanate (FITC), 2′7′-dimethoxy-4′5′-dichloro-6-carbo-xyfluorescein (JOE); rhodamine derivatives such as N,N,N′,N′-tetramethyl-6-carboxyrhodamine (TAMRA), 6-carboxyrhodamine (R6G), tetramethyl-indocarbocyanine (Cy3), tetramethyl-benzindocarbocyanine (Cy3.5), tetramethyl-indodicarbocyanine (Cy5), tetramethyl-indotricarbocyanine (Cy7), 6-carboxy-X-rhodamine (ROX); hexachloro fluorescein (HEX), tetrachlorofluorescein TET; R-phycoerythrin, 4-(4′-dimethylaminophenylaz-o) benzoic acid (DABCYL), and 5-(2′-aminoethyl)aminonaphthalene-1-sulfonic acid (EDANS).

Further FRET donor and acceptor molecules which are particularly suitable for the methods present invention are fluorescent proteins, e.g. dsRed, GFP (Green Fluorescent Protein) or its variants EYFP (Enhanced Yellow Fluorescent Protein), ECFP (Enhanced Cyan Fluorescent Protein), EBFP (Enhanced Blue Fluorescent Protein).

According to another embodiment of the invention, other combinations of donor/acceptor are possible such as fluorescent donor/quenching acceptor or fluorescent donor/fluorescent acceptor, where the emissions can be distinguished by wavelength or lifetime.

Exemplary quencher dyes are well known in the art, e.g. as described by Clegg, “Fluorescence resonance energy transfer and nucleic acids,” Methods of Enzymology, 211:353-389 (1992). Examples of efficient commercially available quenchers are dabcyl, QSY7, QSY9, QSY21, QSY35 (Molecular Probes, Eugene, Oreg.).

The fluorescence donor and acceptor pairs for FRET can be localised on the outside of a particle, on the inside of a particle or can be embedded in the particle membrane or particle shell. In particular embodiments the donor is on the inside of the particle while the acceptor is on the outside or in the wall of the particle or the like. The fluorescence donor and acceptor pairs for energy exchange via FRET can be covalently bound to the particle or can be reversible bound to the particle via ionic interactions or via hydrophilic binding. In particular embodiments the donor and the acceptor are on the inside or on the outside of the particle. The compression and expansion of such a bubble brings donor and acceptor respectively into each other's proximity, or separates them from each other.

In a particular embodiment the fluorescent donor and acceptor molecules are covalently bound to the ultrasound particles or to the compounds used for the manufacture of the compounds. Kits and methods to label biological compounds with organic dyes with dyes are available from e.g. Molecular Probes (Eugene, Oreg., USA). As mentioned above ultrasound particles can be of lipid, carbohydrate or proteinaceous origin (albumin). Products to covalently link proteins (e.g. fluorescent GPF proteins and derivatives) to other proteins lipids or carbohydrates can be obtained from e.g. Pierce (Rockford Ill., USA). The covalent binding allows the binding of a well-determined amount of donor or acceptor to a particle. Alternatively donor and acceptor are labelled separately with the compounds of an ultrasound particle prior to the assembly of such a particle. Labelled and unlabelled amounts can be mixed in a desired amount to achieve a proper spatial distribution of the labels on an ultrasound particle.

In another embodiment, the fluorescence donor and acceptor do not reside on the ultrasound particle. For example, donor and or acceptor molecules are injected whereafter the bubbles take up the dye in the tissue. It is also possible, to inject quenchers or the like. All these chemicals may react with the tissue either to become active or inactive.

In yet another embodiment the fluorescence acceptor and/or donor binds weakly to the ultrasound particle.

In a particular embodiment the particles of the present invention further comprise additional compounds or agents such as compounds or agents for targeting the complete particle to a tissue or a cell type for example via tissue or cell specific bioagents, for example monoclonal or polyclonal antibodies. An example hereof is a particle having antibodies to a bacterium or a virus, allowing the sensitive and specific detection of infections using ultrasound.

In a particular embodiment the particles of the present invention further comprise additional compounds such as bioactive or therapeutically active compounds, e.g. pharmaceutical compounds. These bioactive or therapeutically active compounds can be released from the particles via a passive manner such as diffusion, but can also be released via an active manner for example by increasing the ultrasound frequency and/or amplitude to a level which causes partial or total disruption of the particle.

In a particular embodiment a dye, other than the FRET donor or acceptor, is administered to one or more tissues in the body, before, after or simultaneously with a particle for ultrasound imaging comprising a fluorescence acceptor molecule. If a dye reacts with a certain physiological parameter, such as pH, this parameter can be equally determined using the administered dye. Other metabolic activities that modify or destroy a dye such as oxygen or peroxide or that produces a dye from precursor (e.g. 5′ALA to protoporphyrin) can also by added to the image obtained by the methods and compounds of the present invention. These dyes have been used before in optical (fluorescent) tomography and fluorescence endoscopy.

In a preferred embodiment the dye which is dependent on an environmental condition as mentioned hereabove, is the FRET donor or acceptor on the bubbles of the prevent invention itself, which allows a reduction in the amount of dye needed. However, only parameters that are in equilibrium with the tissue (such as pH, oxygen pressure and temperature) can be measured.

By injection of a dye, the light absorption of the tissue may be changed. This change can be seen in the absorption image. The advantage of the additional dye is that it can have a very different distribution in the body than the bubbles. The bubbles are confined mainly to the vessel system. The dye may be a small molecule that may penetrate cell membranes. The dye may also react with the tissue the change the absorption. An example hereof are pH indicator dyes.

This additional dye may be also a fluorescent dye. With a fluorescent dye, a fluorescence induced fluorescence can occur. One possibility is to have a dye in the tissue that converts the external light to a wavelength that can excite the ultrasound particles to fluorescence. In another embodiment, the administered dye is a fluorescence dye, that can be excited by the fluorescence light of a donor molecule on the ultrasound particle. Again, this additional fluorescence dye may react on external parameters, like pH, temperature and O₂ pressure. If the additional dye is chemoluminiscent, there is no more need for an external light source.

In yet another embodiment chemical (e.g. temperature, pH) sensitive fluorescence donor and acceptor molecules resides on the ultrasound particle.

The dye in the bubbles may react e.g. to pH in a way that allows detection of the presence of the pH environment, and consequently report local acidity or temperature. Suitable pH indicators are active within a pH range of 5,5 to 7,5. This is an option particularly suitable for chemicals that diffuse quickly into the blood stream.

In another aspect the invention relates to a contrast medium such as for ultrasound imaging, comprising particles with fluorescence donors and acceptors for energy exchange via FRET.

Additives for use in a contrast medium are known to the skilled person and include formulations suitable for example for infusion, injection and oral administration such as liquids, sprays and tablets.

For intravascular use, the particles preferably have diameters of less than about 30 micrometer, and more preferably, less than about 12 micrometer. For targeted intravascular use including, for example, binding to certain tissue, such as cancerous tissue, the vesicles can be significantly smaller, for example, less than about 100 nm in diameter. For enteric or gastrointestinal use, the vesicles can be significantly larger, for example, up to a millimeter in size. In general, for medical or diagnostic applications, the vesicles are sized to have diameters of from about 2 micrometer to about 10, 25, 50, 75 or 100 micrometer. The size of the particle can influence their resonant frequency.

According to one embodiment the donor and acceptor molecules for energy exchange via FRET on the particles are within a distance such that no fluorescent light is emitted when the particles are in a resting state. Fluorescent light is emitted upon excitation of the particles with ultrasound and subsequent energy transfer between the fluorescence donor and acceptor molecules. The density of the donor and acceptor molecules on a particle depends from the flexibility of the particle and the type of ultrasound being applied (the density of the dye is higher when the particle is less flexible and the applied ultrasound frequency is lower) and can be determined empirically.

According to another embodiment the fluorescence donor and acceptor molecules on the particles are within a distance such that fluorescent light is emitted when the particles are in a resting state. No or less fluorescent light is emitted upon excitation of the particles with ultrasound and consequently subsequent energy transfer between the fluorescence donor and acceptor for energy exchange via FRET is diminished or abolished.

In another aspect the invention relates to the use of ultrasound for the modulation of fluorescent light emission by particles comprising fluorescence donor and acceptor molecules for energy exchange via FRET. In the method of the present invention an ultrasound energy source is used to force the particle to deform or oscillate which results in a change in distance between fluorescence donor and acceptor molecules which are present on the particle.

Any electromagnetic radiation can be used to excite a fluorescence donor. In the methods of the present invention, excitation of a fluorescence donor can be performed by light of a wavelength of about 160 nm to 2000 nm depending on the particular choice of the fluorochromes.

In another aspect the invention relates to the detection of a modulation of fluorescent light upon subjecting a particle with fluorescence donor and acceptor molecule to ultrasound. Due to the oscillation of the particles, the intensity of the fluorescent light will be continuously switched on and off or modulated. Detection of frequency of oscillation of contrast agents by generation of harmonics is well known in sonography, e.g. Harmonic B-mode imaging as described in “Contrast-enhanced Ultrasound of Liver Diseases”, Solbiati et al., Springer 2003. An aspect of the present invention is to detect such oscillation not by its modulation of ultrasonic energy (or not only by such harmonic ultrasound energy) but by emission or suppression of fluorescence.

FIG. 2 shows a schematic representation of an apparatus which is an embodiment of the present invention. Particles 12 with fluorescent donors and acceptors according to the present invention have been introduced into a sample 13 such as a body organ, a body of a human or animal patient or other object which is to be imaged. The apparatus provides an ordinary B-mode ultrasound image of the body as well as a fluorescence image with a contrast determined by the concentration of said particles 12. For the ultrasound image, a linear ultrasound transducer array 1 transmits an ultrasound pulse of few wavelength as it used for ordinary B-mode imaging with beamlike shape aiming in z-direction. As the pulse travels through the body, reflections on internal surfaces produce an echo signal U(t) received by the transducer 1. The ultrasound receive unit 2 uses the relation z=c*t/2 (c=velocity of propagation in tissue) to transfer this into a 1-dimensional ultrasound image. The pulse emission is repeated with a laterally shifted and/or angulated beam. The ultrasound image reconstruction unit 3 collects the 1-dimensional ultrasound images and calculates a 2-dimensional image from it, that is displayed by the display unit 4.

The fluorescence image is formed parallel to this as described in the following. As the ultrasound pulse traverses the body, it causes oscillations of said particles 12 along its path. One or more optical excitation sources 7 provide excitation light with a spectral overlap of the absorption spectrum of the donor to all parts of the body.

The light sources can be continuous or pulsed, e.g. continuous-wave, modulated or pulsed with defined (variable) wavelength. Acceptors on particles 12 subject to the oscillations produce a fluorescence signal that is proportional to the local concentration of the particles 12 along the path of the pulse. The fluorescent light is detected by a photodiode or an array of photodiodes 8 which are directly attached to the body in order to collect as much of the fluorescent light as possible. Preferable, the diode array covers as much as possible of the body surface for the same purpose. The light input of the photodiodes may be equipped with an optical filter that blocks the light of the optical excitation sources 7 and preferably passes only the fluorescent light so that other, e.g. ambient light does not disturb the signal. The signal detected by the photodiodes are summed and the sum signal S(t) is digitised by an A/D converter 9. Because a useful signal can be recorded only during the first traversal of the ultrasound pulse across the body after its transmission, the operation of the A/D converter 9 is gated by the ultrasound generation unit 2 by means of a gate signal 5. Preferably, the gate signal starts sampling at the time of transmission of the ultrasound pulse and stops sampling after the pulse has either traversed the entire body or after the pulse has been attenuated so much that no useful signal can be recorded any more, whatever time is shorter. These times can be calculated from the size of the body and the attenuation depth of the ultrasound beam. The optical reconstruction unit 10 uses the relation z=c*t to transfer the signal S(t) into a 1-dimensional fluorescent image. In order to improve the resolution along the beam path, the signal can be deconvoluted with the pulse shape of the ultrasound pulse provided by the ultrasound generation unit 2 on a data connection 6. The optical image reconstruction unit 10 collects the 1-dimensional optical images and calculates a 2-dimensional image from it, that is displayed by the display unit 4. The display unit may either display the ultrasound image and the optical image separately or a combination of both, e.g. a color overlay of the optical image to the ultrasound image.

In another embodiment the ultrasound transducer 1 is designed to produce not a beam but a pronounced ultrasound focus at a defined depth and position. By means of the gate line 5 the optical signal is recorded only for the short period of the pulse traversing the focus and the local concentration of the particles 12 at the focus is probed. The focus is stepped across the body probing the concentration point by point instead of a line by line approach. This point approach is slower than the line approach but has the advantage that it is excluded that fluorescence produced by scattered or reflected ultrasound waves may disturb the optical signal as it may be the case in the line approach.

Depending on the settings of the apparatus, different configurations of operation can be envisaged, each of which is an embodiment of the present invention.

Configuration 1: only optical imaging, no combined control, consisting of the following steps:
1. The ultrasound control program starts ultrasound generation.
2. The optical control program starts optical excitation and detection.
3. The optical control program sends the recorded optical data and the information about the scanning sequence to the reconstruction.
4. The ultrasound control program sends the information about the ultrasound generation to the reconstruction.
5. The reconstruction takes optical and ultrasound data and calculates parameters.
6. Display of results, data storage etc.

Configuration 2: optical and ultrasound imaging, no combined control, consisting of the following steps:
1. The ultrasound control program starts ultrasound generation and detection.
2. The optical control program starts optical excitation and detection.
3. The optical control program sends the recorded optical data and the information about the scanning sequence to the reconstruction.
4. The ultrasound control program sends the information about the ultrasound generation and the recorded ultrasound data to the reconstruction.
5. The reconstruction takes optical and ultrasound data and calculates parameters.
6. Display of results, data storage etc.

Configuration 3: optical and ultrasound imaging, combined control, consisting of the following steps:
1. The control program starts ultrasound generation and detection as well as optical excitation and detection.
2. The control program sends the recorded optical data and ultrasound date as well as the information about the scanning sequence to the reconstruction.
3. The reconstruction takes optical and ultrasound data and calculates parameters.
4. Display of results, data storage etc.

Another aspect of the present invention is reconstruction of an image.

One preferred method of reconstruction in accordance with an embodiment of the present invention is iterative reconstruction. It involves a forward model, which is a method of calculating the data of the measurement for a given set of parameters. For iterative reconstruction an update mechanism modifies the parameter set according to the difference between measured data and calculated data. This update can be a back-projection.

Iterative reconstruction uses these two steps in an alternating manner, as indicated in the following steps:
1. Firstly, the parameters of the object are initialized (by a priori knowledge, or alternatively just by a homogeneous value)
2. The forward model is applied to the parameters, i.e. data are calculated from the parameters.
3. The difference between the calculated and the measured data is used to update the parameters.
4. Steps 2 and 3 are repeated until a predefined stopping criterion is met.

There are numerous ways to perform the reconstruction all of which are included within the scope of the present invention, e.g. Arridge and Hebden, Phys Med Biol 1997, 841-853; Arridge, Inverse Problems 1999, R41-R93. One possible disadvantage of the known approaches is that the reconstruction problem can be posed badly. This means that several parameters can be changed simultaneously in a way that there is almost no change in the output signal. Therefore, there can be a lot of ambiguity in the image. The reconstruction algorithms therefore typically use a lot of prior knowledge about the tissue under examination. This decreases the diagnostic value of the image.

To overcome the problem of the prior art, methods of the present invention propose either localized light sources or light detectors inside the object. These are freely set to any position in the tissue thus transposing a badly posed reconstruction problem into a quite well posed one.

In addition to the method steps known in the prior art, the present invention provides particulate bodies such as bubbles that change their fluorescence effectively and/or spectrum by an external applied pressure. The bubbles are introduced into the object under test, e.g. a tissue. Then various acoustic pressure fields are applied. The present invention contemplates that the pressure fields may have very different shapes. One shape which is easy for reconstruction is a focused ultrasound spot, that moves through the tissue under examination. This is effectively a scan using a focussed spot, whereby the pressure wave is just a known modulation in the imaging process, so a lot of different ways are possible. A common feature of the preferred waves is, that, if some superpositions of them are chosen, a “spot” like focussing of the ultrasound energy is generated at many positions (in the order of number of voxels). A wavefront similar to plane waves from different directions and with different frequencies is also included within the scope of the present invention and can be advantageous with respect to the signal to noise ratio in the final image.

In order to exploit the ultrasound information, the processing unit that takes the measured optical data and reconstructs the parameters should also have access to the information about the generated ultrasound wave. This means the “combined optical ultrasound imaging” technique works with a connection from the reconstruction unit to both the optical data recording and the ultrasound generation. This is the minimum connection of both machines.

But preferably both machines are also interfaced on the control side of the systems, i.e. there should be one unit that controls both the optical excitation and detection as well as the ultrasound generation and detection.

Another preferable interface is that the reconstruction unit not only takes the recorded optical data but also uses the recorded ultrasound data. The ultrasound produces oscillation of the bubbles that will generate the FRET effect, but the ultrasound is at the same time preferably used to make an ordinary ultrasound image of the object. This information can be used in the reconstruction of the image in a beneficial way. In the model used for the reconstruction, the fluorescence of the particulate bodies, e.g. bubbles and the (known) pressure waves are added as parameters. The reconstruction provides the concentration of the particles and some optical properties of the tissue. For intrinsic optical properties of the tissue, the method allows for some interaction of the bubble with the tissue.

It is useful to get rid of one unknown quantity in the reconstruction, e.g. the particulate body concentration, such as the bubble concentration. Bubbles are quite easily seen in an ultrasound image, e.g. because they generate harmonics which can be detected as they are at a different frequency. This then known concentration is inserted into the reconstruction algorithm.

In another aspect the invention relates to particles comprising donor and acceptor molecules for energy exchange via FRET for combined optical-ultrasound imaging.

In an embodiment the combined optical-ultrasound imaging of the present invention is performed on the body or parts of a mammalian subject including humans for the purpose of obtaining information about the subject.

In another aspect, the invention relates to a method of deriving image information from an object comprising particles with fluorescence donors and acceptors, said method comprising the steps of subjecting the object to ultrasound and recording a change in fluorescence light emitted by the particles comprising fluorescence donors and acceptors.

In a particular embodiment the invention relates to a method of providing an image of a body part comprising the steps of a) administering to said body part a contrast medium comprising particles with fluorescence donors and acceptors, b) subjecting the body part to ultrasound, and recording a modulation in fluorescent light emitted by the contrast medium.

In yet another aspect the invention relates to the use of particles comprising donor and acceptor molecules for energy exchange via FRET for the manufacture of a diagnostic contrast medium for ultrasound imaging.

In yet another aspect the invention relates to a pharmaceutical composition comprising the particles of the present invention and a pharmaceutically active compound.

In yet another aspect the invention relates to a device comprising an ultrasound source and an apparatus for the detection of fluorescent light.

The present invention is now further demonstrated by the following examples.

### EXAMPLE 1

### Manufacture of Particles With Fluorescence Donor and Acceptor Molecules.

Green Fluorescent Proteins and their derivative are expressed by recombinant DNA technology using commercially available vectors for Clontech (Palo Alto, Calif., USA). Cross-linking of albumin with respectively CFP (Cyan Fluorescent Protein) and YFP (Yellow Fluorescent protein) is performed using the bifunctional agent DSS (disuccinimidyl suberate, Pierce, Rockford Ill., USA) according to the manufacturers instructions. Mixtures of unlabelled albumin, CFP labelled albumin and YFP labelled albumin are used for the manufacture of albumin microshells as described in U.S. Pat. No. 5,855,865. The shells are tested for their ability to emit fluorescent light upon treatment with ultrasound. The ratio of labelled albumin versus unlabelled albumin is decreased when background fluorescence occurs without ultrasound. The ratio of labelled albumin versus unlabelled albumin is increased when no or insufficient fluorescence occurs upon application of ultrasound. This iterative process determines the desired ratio between labelled and unlabelled albumin to achieve an optimal distance between fluorescence donor and acceptor on the particle.

### EXAMPLE 2

### Configuration of an Apparatus for Combined Optical-Ultrasound Imaging.

According to one embodiment of the invention the apparatus for combined ultrasound/optical imaging comprises the following compounds.

A) AN OPTICAL PART, as used for example in a known optical tomography set-up with any suitable light source, e.g. continuous wave, modulated or pulsed with defined wavelength. The wavelength and bandwidth of the light source is preferably matched to the absorption properties of the dyes involved. Preferred properties are efficient excitation of the fluorescent donor while having a low direct excitation of the acceptor. The light source wavelength is preferably well separated from the emission of the donor, and in a wavelength range wherein auto-fluorescence and absorption of the tissue is low as is the case for near-infrared light. Light being produced by the light source is coupled to the object under investigation sequentially at a number of points. This can be done by a light pipe or optical fibres, which are arranged at the circumference of a measurement chamber (e.g. cylindrical) which contains the object. In order to obtain better optical properties, the chamber can optionally be filled with a matching fluid which has similar scattering properties as tissue and which has a low absorption.

The fluorescence emitted by the object is detected simultaneously at several points, for example by optical fibres in the circumference of the measurement chamber with detectors located on the other end of the fibres. The detection can be spectrally and/or time resolved. For example, in a preferred embodiment, the detection of the transmitted excitation light and the fluorescence is performed separately.

The detected light for the different positions of the illumination of the object is the dataset that is needed for the reconstruction of the absorption and scattering coefficients as well as the contrast agent concentration inside the object. These are the parameters, which are associated with the voxels of the object to be reconstructed. The parameters may represent for example, the absorption length, the scattering length, a (fluorescent) dye concentration.

B) AN ULTRASOUND PART: The ultrasound part of the apparatus comprises at least one transducer. In a preferred embodiment, a regular ultrasound imaging device is used.

In order to exploit the ultrasound and optical information to its maximal extent, the processing unit that takes the measured optical data and reconstructs the parameters preferably has access to the information about the generated ultrasound wave. The apparatus for performing the combined optical ultrasound method of the present invention comprises in one embodiment a connection from the reconstruction unit to respectively the optical data recording apparatus and the ultrasound generating apparatus. The connection may be any suitable connection such as a wireless or a wire, cable, or fibre connection. In a preferred embodiment both the optical data recording apparatus and the ultrasound generating apparatus are interfaced on the control side of the systems by the presence and use of a unit that controls both the optical excitation and detection as well as the ultrasound generation and detection. In another preferred embodiment, the reconstruction unit, in addition to recording optical data, also records and utilises the recorded ultrasound data. The image which is obtained by applying ultrasound, equally can be compared with or merged into the image obtained by the optical imaging.

### EXAMPLE 3

FIG. 3 is a schematic representation of a computing system which can be utilized with the methods and in a system according to the present invention. In particular FIG. 3 shows an implementation of Example 2 as a computer based system. All aspects of example 2 are incorporated in example 3, only the relevant differences are discussed below.

A computer system 50 is depicted which may include a video display terminal 14, a data input means such as a keyboard 16, and a graphic user interface indicating means such as a mouse 18. Computer 50 may be implemented as a general purpose computer, e.g. a UNIX workstation or a personal computer or within a dedicated machine.

Computer 50 includes a Central Processing Unit (“CPU”) 15, such as a conventional microprocessor of which a Pentium IV processor supplied by Intel Corp. USA is only an example, and a number of other units interconnected via system bus 22. The computer 50 includes at least one memory. Memory may include any of a variety of data storage devices known to the skilled person such as random-access memory (“RAM”), read-only memory (“ROM”), non-volatile read/write memory such as a hard disc as known to the skilled person. For example, computer 50 may further include random-access memory (“RAM”) 24, read-only memory (“ROM”) 26, as well as an optional display adapter 27 for connecting system bus 22 to the optional video display terminal 14, and an optional input/output (I/O) adapter 29 for connecting peripheral devices (e.g., disk and tape drives 23) to system bus 22. Video display terminal 14 can be the visual output of computer 10, which can be any suitable display device such as a CRT-based video display well-known in the art of computer hardware. However, e.g. with a portable or notebook-based computer, video display terminal 14 can be replaced with a LCD-based or a gas plasma-based flat-panel display. Computer 50 further includes user interface adapter 19 for connecting a keyboard 16, mouse 18, optional speaker 36, as well as allowing outputs to and optional inputs from an ultrasound generator system 20. System 20 is similar to the ultrasound part of Example 2. The generator 20 may be connected through an optional network 40, e.g. a Local Area Network or a wireless connection or network.

The optical system 21 for detecting the variation in light intensity from the body under test may also be connected to bus 22 via a communication adapter 39. System 21 is similar to the optical part of Example 2. Adapter 39 may connect computer 50 to a data network 41 such as a Local or Wide Area network (LAN or WAN) or a wireless connection. The input to computer system 50 from the optical system 21 will typically be the images captured by the optical system. Computer system 50 sends commands to system 21 to direct the illumination from the optical system and to coordinate the optical system 21 with the ultrasound generator system 20.

A parameter control unit 37 of system 20 and/or 21 may also be connected via a communications adapter 38 to the computer 50, e.g. via a connection such as wireless connection or a LAN, etc. Parameter control unit 37 may receive an output value from computer 50 running a computer program in accordance with the present invention or a value representing or derived from such an output value and may be adapted to alter a parameter of system 20 and/or system 21 in response to receipt of, the output value from computer 50.

Computer 50 also may include a graphical user interface that resides within machine-readable media to direct the operation of computer 50. Any suitable machine-readable media may retain the graphical user interface, such as a random access memory (RAM) 24, a read-only memory (ROM) 26, a magnetic diskette, magnetic tape, or optical disk (the last three being located in disk and tape drives 23). Any suitable operating system and associated graphical user interface (e.g. Microsoft Windows) may direct CPU 15. In addition, computer 50 includes a control program 51 which resides within computer memory storage 52. Control program 51 contains instructions that when executed on CPU 15 carry out the operations described with respect to any of the methods of the present invention. In particular the control program may include a program for the reconstruction of an image from the data received from systems 20, 21. The present invention also includes software for reconstruction of an image. In accordance with an embodiment of the present invention the software implements an iterative reconstruction when executed on a processing engine. It involves a forward model, which is a method of calculating the data of the measurement for a given set of parameters. For the iterative reconstruction an update mechanism modifies the parameter set according to the difference between measured data and calculated data. This update can be a back-projection.

Iterative reconstruction uses these two steps in an alternating manner, as indicated in the following steps:
1. Firstly, the parameters of the object are initialized (by a priori knowledge, or alternatively just by a homogeneous value)
2. The forward model is applied to the parameters, i.e. data are calculated from the parameters.
3. The difference between the calculated and the measured data is used to update the parameters.
4. Steps 2 and 3 are repeated until a predefined stopping criterion is met.

There are numerous ways to perform the reconstruction all of which are included within the scope of the present invention, e.g. Arridge and Hebden, Phys Med Biol 1997, 841-853; Arridge, Inverse Problems 1999, R41-R93. To reduce ambiguity in the image, the reconstruction algorithm preferably uses prior knowledge about the tissue under examination. Alternatively, the present invention uses either localized light sources or light detectors inside the object, e.g. tissue to be measured. These are freely set to any position in the tissue thus providing a quite well posed one.

The present invention provides particulate bodies such as bubbles that change their fluorescence effectively and/or spectrum by an external applied pressure. The bubbles are introduced into the object under test, e.g. a tissue. Then various acoustic pressure fields are applied. The present invention contemplates that the pressure fields may have very different shapes. One shape which is easy for reconstruction is a focused ultrasound spot, that moves through the tissue under examination. This is effectively a scan using a focussed spot, whereby the pressure wave is just a known modulation in the imaging process, so a lot of different ways are possible. A common feature of the preferred waves is, that, if some superpositions of them are chosen, a “spot” like focussing of the ultrasound energy is generated at many positions (in the order of number of voxels). A wavefront similar to plane waves from different directions and with different frequencies is also included within the scope of the present invention and can be advantageous with respect to the signal to noise ratio in the final image.

In order to exploit the ultrasound information, the processing unit that has software for taking the measured optical data and reconstructing the parameters and has access to the information about the generated ultrasound wave. This means that the inputs to the reconstruction algorithm are both the optical data recording and the ultrasound generation data.

Preferably software is provided that controls both the optical excitation and detection as well as the ultrasound generation and detection.

Another preferable interface is that the reconstruction algorithm not only takes the recorded optical data but also uses the recorded ultrasound data. The ultrasound produces oscillation of the bubbles that will generate the FRET effect, but the ultrasound is at the same time preferably used to make an ordinary ultrasound image of the object. This information can be used in the reconstruction algorithm for the image in a beneficial way. In the model used for the reconstruction algorithm, the fluorescence of the particulate bodies, e.g. bubbles and the (known) pressure waves are added as parameters. The reconstruction algorithm provides as output the concentration of the particles and some optical properties of the tissue. For intrinsic optical properties of the tissue, the method allows for some interaction of the bubble with the tissue.

The software algorithm preferably gets rid of one unknown quantity in the reconstruction, e.g. the particulate body concentration, such as the bubble concentration. Bubbles are quite easily seen in an ultrasound image, e.g. because they generate harmonics which can be detected as they are at a different frequency. This then known concentration is inserted into the reconstruction algorithm.

Those skilled in the art will appreciate that the hardware represented in FIG. 3 may vary for specific applications. For example, other peripheral devices such as optical disk media, audio adapters, or chip programming devices, such as PAL or EPROM programming devices well-known in the art of computer hardware, and the like may be utilized in addition to or in place of the hardware already described.

In the example depicted in FIG. 3, the computer program product (i.e. control program 51) can reside in computer storage 52. However, it is important that while the present invention has been, and will continue to be, described accordingly, those skilled in the art will appreciate that the mechanisms of the present invention are capable of being distributed as a program product in a variety of forms, and that the present invention applies equally regardless of the particular type of signal bearing media used to actually carry out the distribution. Examples of computer readable signal bearing media include: recordable type and machine readable media such as floppy disks, an optical storage device such as a CD-ROM or a DVD-ROM, a hard disk of a computer, a tape storage device, a memory of a computer, e.g. RAM or ROM. and transmission type media such as digital and analogue communication links.

Other arrangements for accomplishing the objectives of the method and system embodying the invention will be obvious for those skilled in the art. It is to be understood that although preferred embodiments, specific constructions and configurations, have been discussed herein for devices according to the present invention, various changes or modifications in form and detail may be made without departing from the scope and spirit of this invention.

1. An apparatus for combined optical-ultrasound imaging comprising an ultrasound source and a detector for the detection of emitted fluorescent light characterised in further comprising a reconstruction unit for the generation of an image from detected fluorescent light.

2. The apparatus according to claim 1 further comprising a means for synchronising the emission of ultrasound and/or the detection of fluorescent light and/or the generation of an image.

3. The apparatus according to claim 1 further comprising a connection between the reconstruction unit and the detector for the detection of emitted fluorescent light.

4. The apparatus according to claim 1 further comprising a connection between the reconstruction unit and the ultrasound source.

5. The apparatus according to claim 1 further comprising a light source.

6. The apparatus according to claim 1, further comprising a recorder for recording ultrasound.

7. The apparatus according to claim 5 further comprising a control unit for controlling
a) the generation of ultrasound and/or recording of ultrasound with
b) the emission of light by the light source and/or the detection of light recorded.

8. The apparatus according to claim 1 wherein the light source emits light of a continuous-wave, of a modulated wave or of a pulsed wave.

9. The apparatus for ultrasound imaging according to claim 1 wherein the ultrasound source has means for focussing the ultrasound beam to thereby locally modulate light emission from particles with at least a fluorescent acceptor or a fluorescent donor.

10. The apparatus for ultrasound imaging according to claim 1 wherein the ultrasound source has means to generate pulses of sound waves.

11. The apparatus for ultrasound imaging according to claim 1 wherein the ultrasound source has means to generate extended sound waves with varying frequencies and/or varying direction.

12. Use of a particle comprising a fluorescence donor and a fluorescence acceptor in the manufacture of a contrast agent for combined optical-ultrasound imaging.

13. The use according to claim 12 wherein the donor and acceptor are attached to said particle.

14. Use of a particle comprising a fluorescence acceptor and/or a fluorescent donor for the modulation of fluorescent light emission after the application of ultrasound.

15. The use according to claim 14 wherein both acceptor and donor are present on the particle.

16. The use according to claim 14 wherein the fluorescent light emission is generated by FRET.

17. The use according to claim 14 wherein energy transfer is generated by excited state reactions.

18. The use according to claim 14 further comprising recording a change in fluorescence emitted by the particles after application of the ultrasound.

19. A combined optical-ultrasound contrast medium characterised in comprising a particle with a fluorescence donor and/or acceptor wherein said donor or and/or acceptor are attached to the particle.

20. A method for the manufacture of a particle for ultrasound imaging comprising:
contacting said particle or a compound for said particle subsequently or simultaneously with fluorescence donors and/or acceptors, and
reacting a fluorescence donor and/or acceptor molecule with said particle or a compound for said particle.

21. A kit of parts for combined optical-ultrasound imaging comprising an ultrasound source, a monitor for recording fluorescent light and particles having a fluorescence acceptor and/or a fluorescent donor.

22. A pharmaceutical composition comprising particles characterised by fluorescence acceptor and/or a fluorescence donor, said particles further comprising a pharmaceutically active compound.

23. A method of providing an image of a body part of an individual having a contrast medium which comprises particles comprising a fluorescence donor and/or a fluorescence acceptor,
subjecting the body part to ultrasound, and
recording a modulation in fluorescent light emitted by the contrast medium.

24. A computer based apparatus for executing a reconstruction algorithm of an image of an object from data received from an ultrasound source and detected emitted fluorescent light from a contrast medium which comprises particles comprising a fluorescence donor and/or a fluorescence acceptor, the reconstruction algorithm for the generation of the image from detected fluorescent light comprising a pressure dependent fluorescence model of the contrast medium.

25. An apparatus according to claim 24, further comprising means for measuring the concentration of the contrast agent by ultrasound imaging.

26. An apparatus according to claim 24, wherein the ultrasound source emits sound waves that are pulses and focused to one or more lines or one or more spots.

27. A computer based method for executing a reconstruction algorithm of an image of an object from data received from an ultrasound source and detected emitted fluorescent light from a contrast medium which comprises particles comprising a fluorescence donor and/or a fluorescence acceptor, the method comprising reconstructing the image from detected fluorescent light using a pressure dependent fluorescence model of the contrast medium.

28. A method according to claim 27, further comprising measuring the concentration of the contrast agent by ultrasound imaging.

29. A method according to claim 27, wherein the ultrasound source emits sound waves that are pulses and focused to one or more lines or one or more spots.

30. A software product comprising code for execution of claim 27 when executed on a processing engine.

31. A machine readable data storage device storing the software product of claim 30.
